(19) United States
(12) Patent Application Publication
(10) Pub. No.: US 2004/0258756 A1
(43) Pub. Date: Dec. 23, 2004

(54) Novel process

(76) Inventor: Martin John McLoughlin, Stevenage (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/493,325

(22) PCT Filed: Oct. 23, 2002
(86) PCT No.: PCT/GB02/04820

(30) Foreign Application Priority Data
Oct. 25, 2001 (GB) 01256049

(51) Int. Cl. A61K 9/14

(52) U.S. Cl. 424/489

(57) ABSTRACT

The present invention relates to a novel process for preparing crystalline particles of substance which comprises mixing a solution of the substance in a liquid solvent with a liquid anti-solvent for said substance, which liquid anti-solvent is miscible with the liquid solvent, characterised in that the resultant crystalline particles generated are harvested by a process which comprises: (i) mixing a first liquid phase formed from the resultant suspension of crystalline substance in solvent/anti-solvent mixture with a third liquid which is immiscible with said solvent/anti-solvent mixture or a component thereof thereby forming a second liquid phase and which is a non-solvent for the substance such that the substance is segregated into the second liquid phase; (ii) separating the two phases; and (iii) separating the crystalline particles of substance from the second liquid phase.

The present invention relates to a novel process for isolating crystalline particles of a substance.

Crystallisation of substances may be achieved via a procedure which involves dissolving the substance of interest in a liquid solvent, followed by mixing with a liquid anti-solvent for said substance, which anti-solvent is generally miscible with the solvent.

U.S. Pat. No. 5,314,506 (Merck) describes such a process wherein the substance/solvent and anti-solvent are mixed by impinging flows.

WO 00/38811 (Glaxo Group Limited) describes a process for preparing crystalline particles which comprises mixing the solvent and anti-solvent flows in the presence of ultrasound radiation.

WO01/32125 (Glaxo Group Limited) describes a process for preparing crystalline particles which comprises introduction of a stream of substance/solvent tangentially to a stream of anti-solvent in a cylindrical mixing chamber.

A requirement of the above-mentioned solvent/anti-solvent crystallisation approach is that the resultant crystalline particles must be isolated or “harvested” from the suspension in which they are formed. Particle isolation is a crucial stage because any delays in isolation may cause particle size growth if the substance possesses any significant solubility in the supernatent liquor (i.e. the solvent/anti-solvent mixture) in which it is suspended. Such particle growth affects the control of the process and is especially undesirable for small (e.g. micron size) particles such as those suitable for inhalation therapy.

A basic method of harvesting the crystalline particles from the liquid in which they are suspended is by filtration e.g. under vacuum or over-pressure.

However many substances have a tendency to form a hard filter cake formed from agglomerates of particles which do not readily disperse. If this occurs the object of producing particles of controlled particle size (especially of small e.g. micron particle size) is not achieved.

We have now invented a new harvesting method which overcomes or subtantially mitigates the above mentioned disadvantage.

Surprisingly, we have found that by mixing the suspension of crystalline particles in the liquid solvent/anti-solvent mixture in which they were formed with a third liquid which is immiscible with said solvent/anti-solvent mixture or a component thereof and thereby forms a second liquid phase, and which is a non-solvent for the substance, and which wets the drug in preference to the solvent/anti-solvent mixture, the substance may be segregated into the second liquid phase. The solvent/anti-solvent mixture may then be separated from the second liquid phase having the substance now suspended in it and the substance may be separated from the second liquid phase e.g. by filtration. Under these conditions we have found that a very light filter cake is generally formed in which the particles are not agglomerated and which particles may be readily dispersed in a subsequent liquid.

Thus, according to a first aspect of the invention there is provided a process for preparing crystalline particles of substance which comprises mixing a solution of the substance in a liquid solvent with a liquid anti-solvent for said substance, which liquid anti-solvent is miscible with the liquid solvent, characterised in that the resultant crystalline particles generated are harvested by a process which comprises (i) mixing a first liquid phase formed from the resultant suspension of crystalline substance in solvent/anti-solvent mixture with a third liquid which is immiscible with said solvent/anti-solvent mixture or a component thereof thereby forming a second liquid phase and which is a non-solvent for the substance such that the substance is segregated into the second liquid phase (ii) separating the two phases and (iii) separating the crystalline particles of substance from the second liquid phase.

The third liquid should preferably be immiscible with at least the anti-solvent component of the solvent/anti-solvent mixture.

When the third liquid is miscible with the solvent component of the solvent/antisolvent mixture the solvent will partition between the two liquid phases until equilibrium is achieved. This may result in finite solubility of the substance in the second liquid phase and also reduce the density difference between the two layers, therefore, reducing separation efficiency. Further addition of anti-solvent will however effectively reduce the concentration of the undesired solvent component in the second liquid phase, minimising the solubility of the substance and maximising the density difference between the two liquid layers. Thus step (i) may optionally include the step of adding further anti-solvent to the liquids and mixing.

As a consequence of the immiscibility between the 2 liquid phases, mixing in step (i) will preferably be strong enough to ensure that complete wetting of the substance by the third liquid has occurred. Use of a rotary or magnetic stirrer may be suitable for this purpose.

Preferably the volume of third liquid added to the solvent/anti-solvent mixture is significantly smaller than that of the solvent/anti-solvent mixture (e.g. less than 25% of the volume) such that the suspension of substance in the second liquid phase is more concentrated than that formerly in the first liquid phase. This concentration effect results in processing advantages.

The third liquid should be a sufficiently poor solvent for the substance such that no appreciable dissolution of the substance occurs in the second liquid phase such that the particles produced from the crystallisation are stable in size and do not grow at any appreciable rate. In particular it should be a sufficiently poor solvent for the substance such that bridging between particles in any wet filter cake formed prior to drying does not occur to any significant extent.

Preferably the third liquid together with any solvent that have partitioned into the second liquid phase is a poorer solvent for the substance than the solvent/anti-solvent mixture. This results in lesser likelihood of bridging of particles during drying.

Once the substance has been completely wetted, upon standing the liquid phases will separate with the substance preferentially segregated into the second liquid phase. The forces associating the substance with the third liquid may be quite strong, and may be capable of overcoming the gravitational force when the substance is more dense than either liquid phase.

In step (ii) the liquid phases may be separated by decanting the less dense phase off the more dense phase. Alternatively the more dense phase may be funnelled or gravity fed off the less dense phase.

In step (iii) the second liquid phase may be removed from the suspension of substance in second liquid phase to yield particulate substance eg by filtration of centrifugation. As noted above, such filter cakes tend to be light and permit ready re-dispersion in a subsequent liquid. Alternatively in a preferred method of operation the second liquid phase may be removed by evaporation. This method is, however, less suitable if the concentration of any solvent dissolved in the third liquid is appreciable and the solvent is less volatile than the third liquid. Evaporation may require heating depending on the vapour pressure of the third liquid or second liquid phase.

Alternatively the second liquid phase may be extracted into a stream of gaseous phase eg supercritical carbon dioxide under pressurised conditions provided that the substance is not soluble in the gaseous phase. Under these conditions dry particles may be obtained following depressurisation of the apparatus.

In a further alternative embodiment, the third liquid is a gas at room temperature and pressure and is used in the process under pressure as a liquid. In step (iii) separation may be achieved by releasing the pressure to permit vaporisation of the second liquid phase.

Preferably, the third liquid will have a low boiling point such that its separation from the substance may be achieved by exposure to atmospheric pressure or moderate vacuum and ambient or moderately elevated temperature (e.g. up to 40° C.).

Preferably the crystalline particles are produced by a continuous process which comprises mixing a solution of the substance in a liquid solvent with liquid anti-solvent for said substance such that crystalline particles are generated.

For example, a process for preparing crystalline particles of substance comprises mixing in a continuous flow cell in the presence of ultrasonic radiation a flowing solution of the substance in a liquid solvent with a flowing liquid anti-solvent for said substance, which is miscible with the liquid solvent, and collecting the resultant crystalline particles generated.

A feature of the process is that in a steady state the concentration of dissolved substance in the mixing chamber of the flow cell remains approximately constant since the precipitating substance is replaced by the inflow of further solution. This allows the process to be run continuously and reproducibly.

An apparatus for preparing crystalline particles of a substance in this manner comprises:

(i) a first reservoir of said substance dissolved in a liquid solvent;

(ii) a second reservoir of liquid anti-solvent for said substance;

(iii) a mixing chamber having first and second inlet ports and an outlet port;

(iv) means for delivering the contents of the first and second reservoirs to the mixing chamber via the first and second inlet ports respectively at independent controlled flow rate;

(v) a source of ultrasonic radiation located in the vicinity of the first inlet; and

(vi) means for collecting crystalline particles suspended in the liquid discharged from the mixing chamber at the outlet port.

Alternatively a process for preparing crystalline particles of substance comprises (i) admitting a stream of solution of the substance in a liquid solvent and a stream of liquid anti-solvent for said substance which is miscible with the liquid solvent tangentially into a cylindrical mixing chamber having an axial outlet port such that said streams are thereby intimately mixed through formation of a vortex and precipitation of crystalline particles of the substance is thereby caused; and (ii) collecting the resultant crystalline particles suspended in the stream of liquid discharged from the outlet port of the mixing chamber.

Crystalline particles may also be prepared by batch processes. Typically the solvent and substance will be mixed in a vessel and heated to assist solubilisation of the substance, and then the anti-solvent added to induce crystallisation.

The process of the present invention is particularly suitable for preparing particles of substances which are pharmaceutical or carrier substances suitable for inhalation therapy.

Substances suitable for inhalation therapy include substances applied topically to the lung and nose.

Examples of pharmaceutical substances suitable for inhalation therapy include analgesics, e.g., codeine, dihydromorphine, ergotamine, fentanyl or morphine; anginal preparations, e.g., diltiazem; antiallergics, e.g., cromoglycate (e.g. as the sodium salt), ketotifen or nedocromil (e.g. as the sodium salt); antiinfectives e.g., cephalosporins, penicillins, streptomycin, sulphonamides, tetracyclines and pentamidine; antihistamines, e.g., methapyrilene; anti-inflammatories, e.g., beclomethasone (e.g. as the dipropionate ester), fluticasone (e.g. as the propionate), flunisolide, budesonide, rofleponide, mometasone (e.g. as the furoate), ciclesonide or triamcinolone (e.g. as the acetonide); antitussives, e.g., noscapine; bronchodilators, e.g., albuterol (e.g. as the free base or sulphate), salmeterol (e.g. as the xinafoate), ephedrine, adrenaline, fenoterol (e.g. as the hydrobromide), formoterol (e.g. as the fumarate), isoprenaline, metaproterenol, phenylephrine, phenylpropanolamine, pirbuterol (e.g. as the acetate), reproterol (e.g. as the hydrochloride), rimiterol, terbutaline (e.g. as the sulphate), isoetharine, tulobuterol or (-)-4-amino-3,5-dichloro-α-[[[6-[2-(2-pyridinyl)ethoxy] hexyl]methyl] benzenemethanol; PDE4 inhibitors e,g, cilomilast or roflumilast; leukotriene antagonists e.g. montelukast, praniukast and zafirlukast; diuretics, e.g., amiloride; anticholinergics, e.g., ipratropium (e.g. as the bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; and salts, esters and solvates of any of the above. Other examples include 4-hydroxy-7-[2-[[2-[[3-(2-phenylethoxy)propyl]sulfonyl]ethyl] amino]ethyl-2(3H)-benzothiazolone or butixicort and salts and solvates thereof.

Other examples of pharmaceutical substances suitable for inhalation therapy which are of particular interest are: (2R,3R,4S,5R)-2-[6-Amino-2-(1S-hydroxymethyl-2-phenyl-ethylamino)-purin-9-yl]-5-(2-ethyl-2H-tetrazol-5-yl) -tetrahydro-furan-3,4-diol or a salt thereof (eg. the maleate salt); (2S)-3-[4-({[4-(Aminocarbonyl)-1-piperidinyl]carbonyl}oxy)phenyl]-2-[((2S)-4-methyl-2-{[2-(2-methylphenoxy) acetyl]amino}pentanoyl)amino] propanoic acid or a salt thereof (e.g. as free acid or potassium salt); and 6α,9α-Difluoro-17α-[(2-furanylcarbonyl)oxy]-11β-hydroxy-16α-methyl-3-oxo-androsta-1,4-diene-17β-carbothioic acid S-fluoromethyl ester.

Examples of other pharmaceutical substances for which the process according to the invention is useful include compounds to be administered orally such as 2(S)-(2-benzoyl-phenylamino)-3-{4-[2-(5-methyl-2-phenyl-oxazol-4-yl)-ethoxy]-phenyl}-propionic acid, 2,6-diamino-3-(2,3,5-trichlorophenyl)pyrazine and naratriptan (e.g. as hydrochloride). Another compound of interest is (S)-[2-(1-iminoethylamino)ethyl]-L-homocysteine or a salt or racemate thereof (eg. preferably the 2-isomer).

Pharmaceutical substances as described above include asymmetric molecules which may exist as mixtures of optical isomers (e.g. as racemates) or as purified single enantiomers.

Pharmaceutical substances of particular interest include fluticasone, beclomethasone, salmeterol, salbutamol or an ester, salt or solvate thereof. The substance of most interest is salmeterol xinafoate (including the racemate or the purified r- or s- enantiomers). Fluticasone propionate is also of particular interest.

Examples of carrier substances include lactose.

The solvent and anti-solvent liquids will be selected so as to be appropriate for the substance. They should be readily miscible in the proportions employed. Suitable combinations of solvent/antisolvent include acetone/water, ethanol/IPA, methanol/IPA, methanol/water and reciprocal pairs. Methanol/IPE is also a suitable pairing. As noted above the third liquid will be selected so as to be immiscible with the solvent/anti-solvent mixture or a component thereof and so as to be a non-solvent for the substance.

The solvent and anti-solvent may each be mixtures of liquids if desired or necessary.

For generation of small particles by the process according to the invention, it is preferred that the difference between the dissolution properties of the solvent and anti-solvent be as great as possible. For reasons of industrial efficiency (particularly in order to reduce the throughput volumes of liquid) it is preferred to use concentrations of substance in solvent which are as high as possible. Nevertheless the solutions must be stable and not prone to crystallisation before discharge into the continuous flow cell. With this end in mind, it may be preferred to use the solution of the substance in the solvent at elevated temperature. It may also be preferable to cool the anti-solvent.

In order to prevent premature precipitation of the dissolved substance in the lines it will generally be desired to prime the apparatus by first pumping it with solvent. It may be preferred to prime the apparatus by pumping it with heated solvent, particularly when the dissolved substance is close to its solubility limit.

When the substance is fluticasone propionate we prefer the solvent to be acetone and the anti-solvent to be water. Preferably the third liquid is a lower alkane which is a liquid at ambient temperature and pressure e.g. hexane, pentane, heptane or iso-octane, especially n-hexane. A lower alkane which is liquified under pressure e.g. butane may also be employed. Ethers may also be suitable.

When the substance is salmeterol xinafoate we prefer the solvent to be methanol or acetone (more preferably methanol) and the anti-solvent to be water. Preferably the third liquid is a lower alkane which is a liquid at ambient temperature and pressure eg hexane, pentane, heptane or iso-octane, especially n-hexane. A lower alkane which is liquified under pressure e.g. butane may also be employed.

When the substance is salbutamol sulphate we prefer the solvent to be water and the anti-solvent to be IMS. When the substance is beclomethasone dipropionate we prefer the solvent to be IMS and the anti-solvent to be water. When the substance is lactose we prefer the solvent to be water and the anti-solvent to be ethanol. When the substance is budesonide, we prefer the solvent to be methanol and the anti-solvent to be water. When the substance is formoterol fumarate or terbutaline sulphate we prefer the solvent to be methanol or acetone and the anti-solvent to be water. When the substance is 2,6-diamino-3-(2,3,5-trichlorophenyl)pyrazine we prefer the solvent to be methanol and the anti-solvent to be water.

When the substance is 2(S)-(2-benzoyl-phenylamino)-3-{4-[2-(5-methyl-2-phenyl-oxazol-4-yl)-ethoxy]-phenyl}-propionic acid we prefer the solvent to be acetone and the anti-solvent to be water. When the substance is naratriptan hydrochloride we prefer the solvent to be methanol and the anti-solvent to be IPE. When the substance is 6α, 9α-difluoro-11β-hydroxy-16α-methyl-3-oxo-17α-propionyloxy-androsta-1,4-diene-17β-carbothioic acid S-(2-oxo-tetrahydro-furan-3-yl) ester we prefer the solvent to be acetone and the anti-solvent to be water.

The preferred third liquid is a lower alkane which is a liquid at ambient temperature and pressure eg hexane, pentane, heptane or iso-octane, especially n-hexane. The preferred anti-solvent is immiscible with such a third liquid and the preferred solvent, which will be selected accordingly, will preferably be immiscible with such a third liquid.

We have found that the method according to the invention is suitable for producing populations of mixtures when the substance is a mixture of substances. When the substance is a mixture the method has particular advantages since it is capable of producing mixtures of crystalline particles of very high homogeneity without the need for any blending step. When the substance is a mixture the solvent and anti-solvent will have to be appropriate for all components of the mixture. Differential solubilities in the recrystalline mixture tend to result in the output proportions of the mixture differing from the initial proportions in solution in the solvent and so appropriate adjustment of the input proportions to achieve the desired output proportions may be necessary.

The method according to the invention is particularly suitable for producing mixtures of crystalline particles of salmeterol and fluticasone or salts and esters thereof e.g. salmeterol xinafoate and fluticasone propionate. The preferred solvent is acetone. The preferred anti-solvent is water. The preferred third liquid is a lower alkane which is a liquid at ambient temperature and pressure eg hexane, pentane, heptane or iso-octane, especially n-hexane Recrystallisation from acetone using water as anti-solvent tends to cause an increase in the ratio of salmeterol xinafoate to fluticasone propionate relative to their proportion in solution in acetone. The method is also expected to be suitable for producing mixtures of crystalline particles of formoterol and budesonide or salts and esters thereof e.g. formoterol fumarate and budesonide.

As a further aspect of the invention we provide a population of particles obtainable by a process according to the invention.

Particles of pharmaceutical or carrier substances may be obtained which are suitable for use in a pharmaceutical composition for inhalation therapy, such as dry powder composition (whether containing pure drug, or drug mixed with a carrier such as lactose) or a pressurised liquid formulation (e.g. a formulation comprising a hydrofluoroalkane (HFA) propellant such as HFA134a or HFA227 or a mixture thereof.

Pressurised liquid formulations suitable for metered-dose inhalers will be retained in canisters, typically aluminium canisters (which may be plastics lined) which are provided with a metering valve of appropriate metering volume.

It will be appreciated that references to inhalation therapy also extend to administration of pharmaceutical compositions via the nasal route. Formulations suitable for nasal delivery include pressurised (e.g. HFA containing) formulations and non pressurised (e.g. aqueous) formulations which may be metered by the delivery device adapted for administration to the nose.

We also provide a pharmaceutical composition comprising a population of particles prepared according to the invention.

Apparatus suitable for use in the present invention is illustrated by reference to FIG. 1 in which mixing chamber 1 is provided with first inlet port 2 connected to first reservoir 3 containing substance dissolved in solvent and second inlet port 4 connected to second reservoir 5 containing anti-solvent. Pumps 6 and 7 deliver liquid from reservoirs 3 and 5 to mixing chamber 1 at a controlled rate. An ultrasound probe 8 is located in the vicinity of, and just above, inlet port 2. When pumps 6 and 7 are in operation, liquids from reservoirs 3 and 5 are delivered to mixing chamber 1 and are mixed with the aid of magnetic stirrer 9. Liquid containing the particles of substance thus generated flows out of the mixing chamber via exit port 10. Details not shown: this suspension may be mixed with the third liquid, vigorously mixed, and the second phase containing third liquid and substance decanted or funnelled off. The substance may be harvested from the liquid phase by fltration followed by drying.

### BRIEF DESCRIPTION OF THE DRAWINGS

FIG. 1 shows example apparatus suitable for use according to the invention.

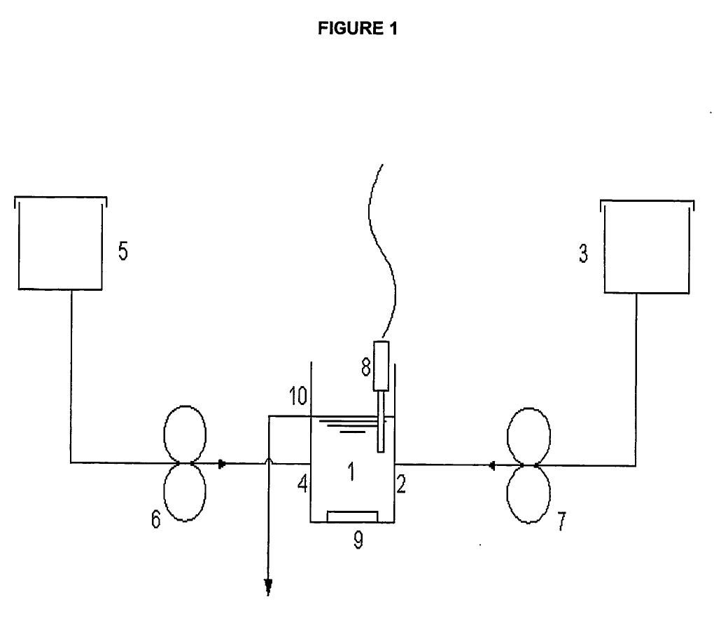

The present invention is further illustrated by the following Examples:

### EXAMPLE 1

### Isolation of fluticasone propionate

Fluticasone propionate is not wetted by water nor is it soluble in water (i.e. it is hydrophobic in nature). However, fluticasone propionate is readily soluble in acetone. Therefore, fluticasone propionate was crystallised in accordance with the process described in WO 00/38811, wherein 30 g of fluticasone propionate was yielded from a 1:4 w/w ratio of solvent :anti-solvent (acetone:water). The resultant liquor composition comprised >60% w/w water and as a consequence fluticasone propionate was insoluble in the resultant composition. The resultant liquor composition was added to a 5L duran flask containing between 50 and 150 ml hexane as third liquid and 2L of distilled water (which was added to reduce the acetone concentration in the alkane layer by partitioning and increasing the density difference between the liquid layers, resulting in improved separation efficiency). The addition of the slurry immediately resulted in the formation of two liquid layers, the uppermost being hexane/acetone and the lower being water/acetone.

The duran flask was then vigorously shaken to break the interface between the layers causing full wetting of the fluticasone propionate by the alkane. The duran flask was then left to stand wherein after 5 minutes it was observed that most of the fluticasone propionate had segregated into the alkane layer. Complete segregation was achieved within 12 minutes and the upper layer containing substance was decanted off. The substance was separated from the liquid phase and dried by filtration under vacuum overnight. The resultant particles were light, low density and readily dispersed.

### EXAMPLE 2

### Isolation of salmeterol xinafoate

20 ml of a solution of 40 mg/ml salmeterol xinafoate in methanol was precipitated by addition to 180 ml of de-mineralised water at room temperature, using a syringe. The salmeterol xinafoate was rapidly precipitated to form a milky, opaque suspension of salmeterol xinafoate in water-methanol. The suspension showed little tendency to settle, indicating relatively fine particles. Subsequently 20 ml of hexane was added to the suspension which was then shaken vigorously. On settling, the salmeterol xinafoate flocculated and extensively partitioned into the hexane layer. Further vigorous shaking and subsequent standing resulted in almost all of the salmeterol xinafoate partitioning into the hexane layer. The hexane layer was decanted from the methanol-water layer and vacuum filtered until dry.

The patent applications cited in this specification are hereby incorporated by reference.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

1. A process for preparing crystalline particles of substance which comprises mixing a solution of the substance in a liquid solvent with a liquid anti-solvent for said substance, which liquid anti-solvent is miscible with the liquid solvent, characterised in that the resultant crystalline particles generated are harvested by a process which comprises:
(i) mixing a first liquid phase formed from the resultant suspension of crystalline substance in solvent/anti-solvent mixture with a third liquid which is immiscible with said solvent/anti-solvent mixture or a component thereof thereby forming a second liquid phase and which is a non-solvent for the substance such that the substance is segregated into the second liquid phase;
(ii) separating the two phases; and
(iii) separating the crystalline particles of substance from the second liquid phase.

2. A process according to claim 1 wherein the mixing of a solution of the substance in a liquid solvent with a liquid anti-solvent for said substance is carried out in a continuous flow cell in the presence of ultrasonic radiation.

3. A process according to claim 1 in which the third liquid is immiscible with at least the anti-solvent component of the solvent/anti-solvent mixture.

4. A process according to claim 1 in which the volume of the third liquid added to the solvent/anti-solvent mixture is less than 25% of the total volume.

5. A process according to claim 1 in which the third liquid is a lower alkane which is a liquid at ambient temperature and pressure.

6. A process according to claim 5 in which the lower alkane is n-hexane.

7. A process according to claim 1 in which in step (ii) the liquid phases are separated by decanting the less dense phase off the more dense phase.

8. A process according to claim 1 in which in step (iii) the crystalline particles of substance are separated from the second liquid phase by evaporation.

9. A process according to claim 1 in which in step (iii) the crystalline particles of substance are separated from the second liquid phase by extraction into a supercritical fluid.

10. A process according to claim 1 in which the substance is a pharmaceutical suitable for inhalation therapy.

11. A process according to claim 10 in which the substance is fluticasone, beclomethasone, salmeterol, salbutamol or an ester, salt or solvate thereof.

12. A process according to claim 11 wherein the substance is fluticasone propionate.

13. A process according to claim 11 wherein the substance is salmeterol xinafoate.

14. A process according to claim 1 wherein the substance is a mixture.

15. A process according to claim 14 wherein the substance is a mixture of fluticasone propionate and salmeterol xinafoate.

16. A process according to claim 12 wherein the solvent is acetone and the anti-solvent is water.

17. A process according to claim 13 wherein the solvent is methanol and the anti-solvent is water.

18. A population of particles obtainable by a process according to claim 1.

19. A pharmaceutical composition comprising a population of particles according to claim 18.

20. A process according to claim 15 wherein the solvent is acetone and the anti-solvent is water.
